(19) United States
(12) Patent Application Publication
(10) Pub. No.: US 2005/0197651 A1
(43) Pub. Date: Sep. 8, 2005

(54) Vaginal ring preparation and its application

(76) Inventors: Hai Lin Chen, Shanghai (CN); Hai Hao Shao, Shanghai (CN); Jian Xing Chen, Shanghai (CN); Liang Kang Chen, Shanghai (CN)

Correspondence Address: Joseph L. Strabala, Esq.;Law Office of Joseph L. Strabala, Suite 1020, One Embarcadero Center, San Francisco, CA, 94111, US

(21) Appl. No.: 11/072,756

(22) Filed: Mar. 4, 2005

Related U.S. Application Data
(63) Continuation-in-part of application No. 10/134,402, filed on Apr. 25, 2002, now abandoned.

(30) Foreign Application Priority Data
Apr. 25, 2001 (CN) 01112712.0

(51) Int. Cl. A61K 31/56; A61K 9/22

(52) U.S. Cl. 604/890.1; 514/170

(57) ABSTRACT

A vaginal drug delivery device includes a tubular base of an inert rubber composition, a first layer having a thickness up to 3 mm composed of a mixture of a drug to delivered, at least one surfactant and at least one dispersing agent applied to said outer surface of the tubular base, and a second layer of silicone rubber having a thickness up to 1 mm encapsulating the first layer on the tubular base whereby said drug will diffuse through said second layer when the device is inserted into the vagina to treat the patient with the drug in the first layer.

This applicaition is a continuation in part of application Ser. No. 10/134,402 filed on Mar. 4, 2002 and claims priority on application number 01112712.0 of the People's Republic of China dated Apr. 25, 2001.

### BACKGROUND

This invention of vaginal insert is a new method in terms of the preparation method and its application.

Mifepristone [Miteprex®, a trademark of Danco Investors Group LP.,] is an anti-progesterone at the receptor level. It has the functions of terminating early pregnancy, interfering implantation, inducing menstruation, and promoting the mature of uterine cervix. It can compete with progesterone in achieving the effect of anti-progesterone. It also has certain ability of combining with glucocorticoid receptors. According to clinical experiments, Mifepristone can combine with the estrogen and androgen receptors, reducing the available receptors that can be combined with estrogen and androgen in uterine myoma tissue. It is used widely in clinic for the treatment of uterine myoma and uterine endometriosis. At the same time, it can be used for contraceptive purpose.

However, the biological effectiveness is low if it is used orally. In order to reach certain treatment regimes, the oral dose has to be increased to 10-25 mg/day and used for several months or years. When this drug combines with glucocorticoid receptors, patients feel fatigue, nausea, dizziness, vomiting and other gastrointestinal side effects. When used to treat uterine myoma and uterine endometriosis, this drug has to be used for a long time but its side effects make it hard for patients to do so and limit this medicine in reaching its full benefits.

Danazol (generic name) is a synthesized derivative of 17-methyl testosterone. It has similar effects as testosterone and can inhibit the function of ovary by inhibiting the secretion of luteinizing hormone by corpus leteum. It also can be used for the treatment of endometriosis and uterine endocrine diseases. It can also be used to shrink uterine myoma.

However, the doses needed for such purposes for this drug are large, 400-800 mg per day, for more than 6 months, which costs a lot of money. Also the side effects of masculine like changes, including acne, hirsutism, tense voice and body weight gain, are very common.

Danazol also inhibits the ovary function and lowers the level of estrogen below a normal level. The drug also induces menopause symptoms, such as sweating, palpitation, anxiety, which are unacceptable for women.

The current invention reduces some of these side effects by a improved and more reliable delivery system for Danazol and other drugs in the vaginal cavity.

For example, Progesterone can be used to treat the early signs of abortion, functional uterine bleeding, uterine endometriosis, uterine endometrium adenoma but is useless if taken orally. It is typically taken by injection or the like, which is often not acceptable for patients who need to take this medicine for an extended period of time.

Also, selective estrogen antagonists, such as Raloxifene (generic name) and Tamoxifene (generic name), need large doses for a long period of time when used to treat endometriosis.

In order to treat uterine myoma, and uterine endometriosis, more effectively, patients need methods which are more convenient with less side effects and more apparent treatment effects.

In 1970, the idea of vaginal ring was proposed by Mishell and was used in clinical trial. Later, E2-ring, Levonorgestetrel vaginal ring, was invented for contraceptive purpose, hormone-replacing therapy in treating gynecological diseases However, the drugs released from this kind of vaginal ring were typically less than 150 μg per day which release decreased rapidly over time, see e.g., graph, FIG. 8 of U.S. Pat. No. 3,920,805 issued to Roseman. The Levonorgestetrel technique was not able to produce a vaginal ring that could release large quantity (selected dosage) of insoluble drugs over an extended treatment period with a more or less constant dosage.

There are a lot of vaginal drug delivery products in the world similar to those described by Levonorgestetrel but they are ineffective to release insoluble drugs in a desired quantity (dosage), more or less at a constant rate for an extended period of time.

The main purpose of this invention is to provide a constant, stable drug releasing system, like this vaginal ring, that can release generally insoluble drugs in a large enough quantity, over time, to be medically effective. Also the invention provides a new treatment regime, in which vaginal rings and the like can be used.

In the past generally insoluble drugs are distributed molecularly in the dispenser. They employ special contacting areas between the drugs being released and the releasing media, which increase the solubility of the drugs by increasing the surface area.

In such dispensers, the drugs are distributed molecularly in the dispensers and mechanically mixed with silicone or high molecular substances, whereby the solubility of the drugs is impacted because of the interstitial characteristics of these high molecular substances.

According to this invention surface active agents are added to the drugs to improve the delivery of such drugs by increasing their solubility. Moreover these surface-active agents are more effective as the temperature of the novel dispenser is increased, such as when the device us placed in the uterine cavity. The surface active agents operate by increasing the mechanical space and creating canal structures within the silicone rubber or high molecular materials which makes drugs more soluble.

The biological availability of the drugs is improved because of their increased solubility (concentration) due to the addition of surface-active agents.

This invention is achieved by using a dispenser, such as vaginal ring, on which the drugs mixed with surface active agents are placed with a silicone rubber sheet covering the mixture.

The medicines [drugs] account for 5-7% of total weight. The physiologically acceptable surface active agents account for 0.5-20% of total weight. The balance of the weight are contributed by the physiologically acceptable dispensers and the silicone layer, which covers the medicines, with a layer which is 0.02-1 mm. in thickness.

The medicines that can be delivered by by this novel dispenser are Mifepristone, Danazole, Progesterone, and one or several selective estrogen antagonists, including Raloxifene, Tamoxifene and Nafoxidine along with other drugs.

The physiologically acceptable surface active agents are selected from one or more mixtures of Span 20-80, Brij 52-76, OP emulsifiers, PEG 400-20000, Pluronic-124, Pluronic-1 88, Sodium lauryl sulphate, Sodium teradecyl sulpahte, Trolamine (generic name).

The physically acceptable dispersing agents mentioned above are selected from one or more mixtures of Glycerin, Methylethylene glycol, PEG 400-20000, Succinic acid, Cholic acid, Deoxycholic acid, Hexadecyl alcohol, Octadecul alcohol, B Type cyclodextrin, R Type cyclodextrin and silicone rubber. If one of the surface-active agents is PGE or Brij, the dispersing agents should be different from the surface active agents.

The silicone rubbers are selected from HTV, RTV-2, RTV-1 or LTV, Dow Corning Medical Silicone Rubber Silastic-382 made in USA, Dow Corning Q7 Medical Silicone Rubber series made in USA, Dow Corning Implantable MDX series silicone rubber made in USA, or other medical silicone rubber.

The silicone rubber covering the medicines are obtained from HTV (RT silicon rubber with molecular weight of 0.3-1 million), RTV-2 (double composition RT silicon rubber with molecular weight of 0.0074-0.11 million), RTV-1 (single composition RT silicon rubber with molecular weight of 0.0074-0.11 million) or L TV (RT silicon rubber with molecular weight of 400-20000), Dow Corning Medical Silicone Rubber Silastic-382 made in USA, Dow Corning Q7 Medical Silicone Rubber series made in USA, Dow Corning Implantable MDX series silicone rubber made in USA, or other medical silicone rubber.

The medicines are contained in the center of vaginal ring and are surrounded by internal tubes, which could be made from medical silicone rubber. The center of the vaginal ring is empty. The thickness of the silicone rubber covering drugs and agents is 0.02-mm.

### SUMMARY OF THE INVENTION

A vaginal drug delivery device includes a tubular base of an inert rubber composition, a first layer having a thickness up to 3 mm composed of a mixture of a drug to delivered, at least one surfactant and at least one dispersing agent applied to said outer surface of the tubular base, and a second layer of silicone rubber having a thickness up to 1 mm encapsulating the first layer on the tubular base whereby said drug will diffuse through said second layer when the device is inserted into the vagina to treat the patient with the drug disposed in the first layer.

A preferred design for the tubular base is a ring.

### DESCRIPTION OF THE DRAWINGS

FIG. 1 is an illustration of a vaginal ring which can be employed in this invention;

FIG. 2a is the cross section of the vaginal ring shown in FIG. 1 employed in one application of the invention;

FIG. 2b is the cross section of the vaginal ring employed in another application of the invention;

FIG. 2c is the cross section of the vaginal ring employed in the third application of the invention;

FIG. 3 is a graph showing the delivery rates of Mifestone in prior art delivery devices;

FIG. 4 is a graph showing the improved delivery rates of Misfestone employing this invention;

FIG. 5 is a graph showing the delivery rates of Danazol in prior art delivery devices; and

FIG. 6 is a graph sowing the improved delivery rates of Danazol employing this invention.

The following are the detailed description of this invention using these drawings as illustrations.

In FIG. 1, the diameter of the vaginal ring is 1-10 cm.

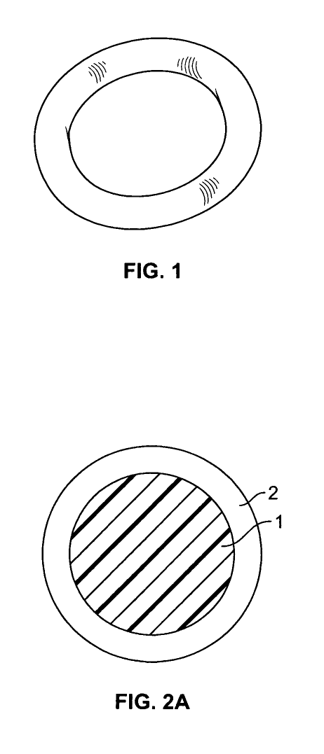

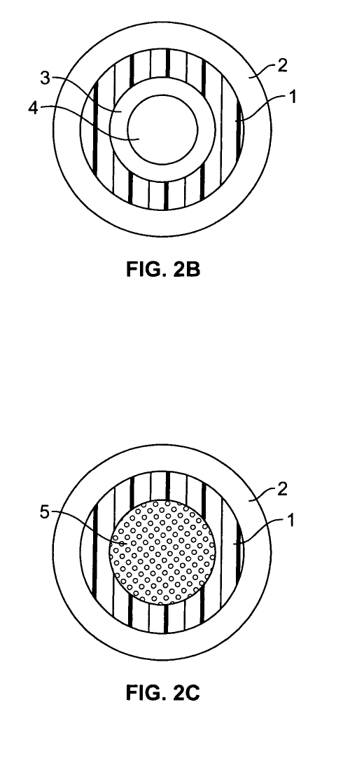

In FIG. 2a, part 1 (shadowed part) refers to the part containing medicines. Part 2 refers to the silicone rubber cover, which is 0.02-1 mm in thickness.

In FIG. 2b, Part 1 (shadowed part) refers to the part containing medicines. Part 2 refers to the silicone rubber covering, Part 3 refers to the internal tubes, which are made of medical silicone rubber or other high molecular polymers. These internal tubes surround part 4, which is the empty core with diameter of 2.5-6.5 cm. The thickness of internal tube is 1-6 mm. The thickness of drugs and agents is 0.5-3 mm. The thickness of the silicone rubber covering is 0.02-1 mm.

### DESCRIPTION OF A PREFERRED EMBODIMENT

The techniques used to produce this vaginal ring are some advanced techniques in this area, including injection molding process, mold molding process, extrusion molding process, wetting process, etc. The following steps are included:

A. Prepare the medicine part based on the concentrations mentioned above. Then put the prepared drugs (mixture) into the medical silicone rubber tubes and put the tubes in the molding machine to form certain shape by heating or under pressure.

Or

B. (1) Prepare the silicone rubber tube to solid cylinder with the required size, which is the medical silicone rubber cylinder.
(2) Prepare the drugs according to the concentrations mentioned above and make them into thin layers.
(3) Cover the medical silicone rubber cylinder mentioned in (1) with the thin layers mentioned in (2),
(4) Cover the products got from (3) with another layer of medical silicone rubber with a the thickness of 0.02-1 mm.
   Or
C. (1) Choose the internal tubes that are made from medical silicone rubber or other chemical substances with diameter of 2.5-6.5 cm.
(2) Prepare the drugs based on the concentrations mentioned above and make them into thin layers.
(3) Cover the internal tubes with the thin layers from step (2).
(4) Cover another layer of medical silicone rubber with thickness of 0.02-1 mm.
   Or
D. Put 1 g. of silicone rubber into organic solvent, such as petroleum ether. Dip products from B(3) or C(3) into this solution for 5 seconds and remove them and dry them.

This illustrated vaginal ring can be used to treat uterine myoma, uterine endometriosis and other related diseases of women. This product could also be used for contraceptive purpose. This vaginal ring can release drugs, at a more of less constant rate and in larger dosages than previous devices. The amount of drugs released can reach 1-10 mg per day. Also it has minor side effects compared to other delivery methods.

The following are some further descriptions of this novel dispenser in some clinical applications.

Application 1:

Mix 2.1 g progesterone, 0.1 g Sodium lauryl sulphate, 01 g Span-20, 0.7 9 beta cyclodextrin (molecular weight is 1134, produced by Shanghai Testing Agents Company). Fill the mixture into the medical silicone rubber tube (Silastic-382, thickness is 1 mm). Then mold them into desire shape in the molding machines by heating.

Application 2:

Use an extrusion molding process to make a medical silicone rubber ring with 5 cm diameter with HTV medical silicone rubber. Mix 0.15 9 Raloxifene, 0.01 Sg Brij 52 and 2.835 HTV medical silicone rubber (molecular weight is 0.3-1 million, produced by Shanghai Rubber Research Institute). Mold the mixture into thin layers (thickness is 1 mm). Then cover the medical silicone rubber rings with these thin layers. Cover this layer with another layer of HTV medical silicone rubber (thickness is 0.02 mm) and mold them into desired shape with the molding machine.

Application 3:

Using an extrusion molding process produce the medical silicone rubber internal tube (made from R TV-1, produced by Shanghai Rubber Institute) with diameter of 4 mm. Then make the silicone rubber tubes into shape of circle with diameter of 5 mm.

Mix 0.3 g Mifepristone, 0.6 g Sodium lauryl sulphate, 0.3 Span-80 and 1.8 PEG 1200. Turn the mixture into thin layers. Then make thin layers with thickness of 0.02 mm from RTV-1 medical silicone rubber and cover these internal rubbers with these thin layers. Mold the final product into desired shape under heating.

Application 4:

Put 9 L TV (produced by Shanghai Rubber Research Institute) into 20 ml Petroleum ether and mix them together as dipping solution. Mold the HTV medical silicone rubber (molecular weight is 0.3-1 million (made by Shanghai Rubber Research Institute) into the shape of ring with diameter of 4 cm. Mix 1.5 Danazol, 0.03 Sodium lauryl sulphate and 1.47 PEG 20000 and make them into thin layers. Cover the medical silicone rubber rings with these thin layers and put the rings into the dipping solution above for 5 seconds. Then remove them and dry.

Comparison studies on these application cases:

Drug Releasing Test:

Devices used: 1. High Pressure Liquid Chromatography (HPLC). Shimadzu LC-1 OA T

2. Shaking water bath: HZS-H
   Process:
1. Tie the vaginal ring into a big test tube (125 ml). Add 100 ml distilled water into the tube under 37C. Shake the testing tube for 24 hours with shaking rate of 60/minute. Then take the vaginal ring out.
2. The standard sample is the Mifepristone provided by Shanghai Family Planning Institute.
3. C-18-250 nm Analytic column (Brand: Shimadzu): Pre-set HPLC. The testing conditions are:
   Wavelength 310 nm.
   Sensitivity: 0.1 AUFS
   Speed: 3 Atten. 5
   Flow phase: methanol: water=70:30
   Moving speed=1 ml/minute
   Injection amount: 10 μl
   Peak time: 5.48 minutes

Take 23 μl/ml standard sample, 10 μl injection sample to get the peak area of 656504-656800.

Prepare the calibration based on the amount contained in 100 ml.

Results: The releasing amount in application 2 is: 1-2 mg/day

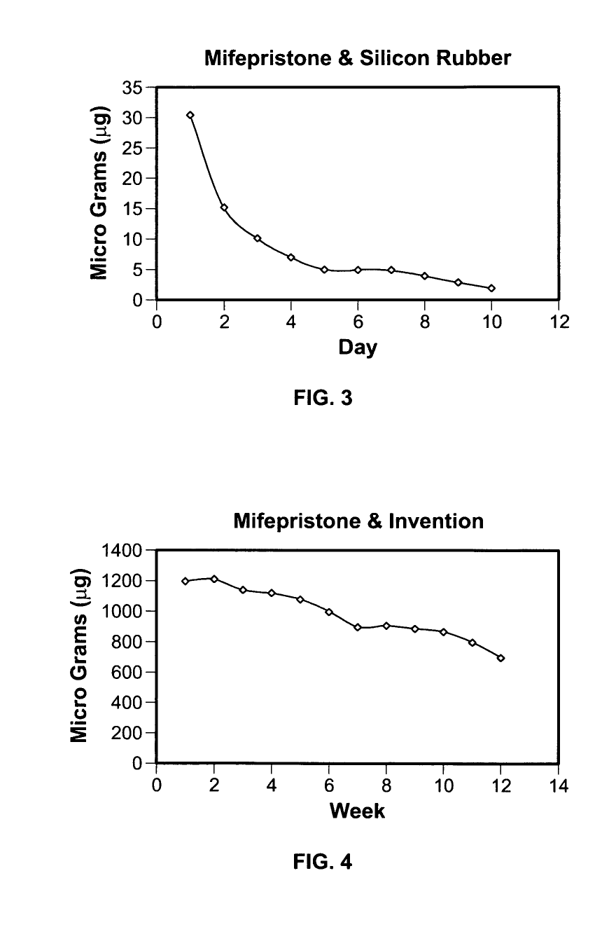

Referring to FIG. 3, the graph shows the delivery rate of Mifestone in a prior art vaginal ring with the vertical or y-axis, showing the concentration of the drug released, over days shown on the x-axis. This release profile is consistent with the information in the prior art, see e.g. U.S. Pat. No. 3,920,805 issued to Roseman.

In contrast using the teachings of the current invention, the delivery rate of Mifestone was significantly higher over a longer period of time, as shown in the graph FIG. 4.

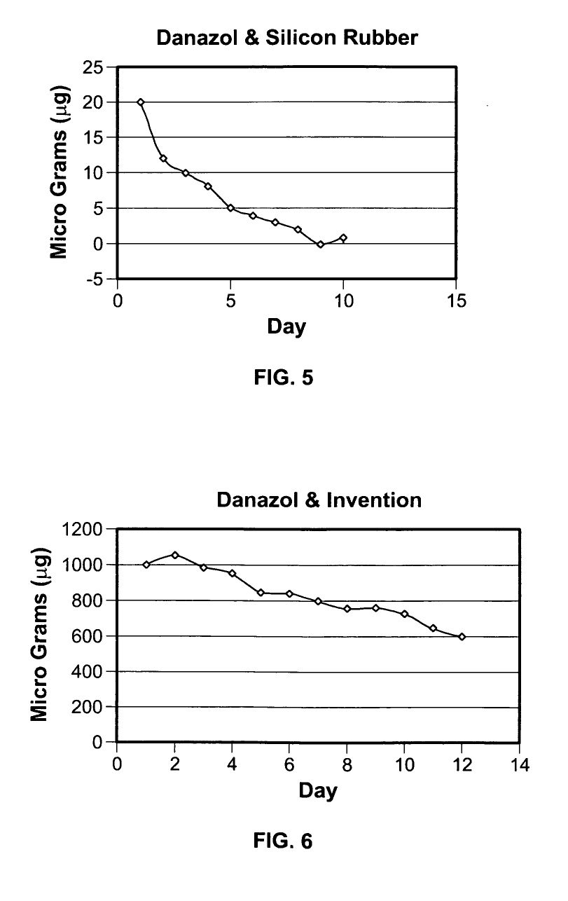

A second comparison of the improved drug delivery rates employing the current invention is found in a comparison of FIGS. 5 and 6. In FIG. 5 the delivery rate over days for Danazol is illustrated employing the prior art devices, while in FIG. 6, the higher delivery rate of this drug over a longer period of time, when using the current invention, is illustrated.

Clinical data suggests that employing the device of this invention to deliver higher dosages drugs in the vaginal cavity over a longer period can be very beneficial for patients.

Clinical Test I:

One female, 47 years old, had abnormal menstruations for the last two years. The amount of bleeding for each period was much more than normal and the period was irregular, with severe abdominal pain. She was diagnosed with uterine myoma in August 2000. In November 2000, it was found that the tumor greatly enlarged.

The patient began to use the vaginal ring mentioned in Application 3 on Nov. 13, 2000. Her menstruation came back on Mar. 20, 2001, which means that this vaginal ring had been releasing effective amount of drugs during the treatment period. She did not have abdominal pain and other discomfort feeling when the menstruation came back. The vaginal ring was removed on March 2001 and the uterus and myoma were found to be much smaller at that time.

Clinical Test 2:

A patient, 48 years old, was diagnosed with endometriosis and uterine myoma. She tried different kinds of treatment but could not achieve beneficial results. She had severe abdominal pain before and after menstruation and had to take analgesics to reduce the pain. In July 2000, she began to use the vaginal ring described in Application 3. After 10 days, pain disappeared. The menstruation came back in October 2000. A new ring was used after her menstruation. In January 2001, it was found that the myoma disappeared. Patient's self-feeling was quite good.

1. A vaginal controlled drug delivery device comprising:
a tubular base of an inert rubber composition having an outer surface;
a mixture comprising a drug to be delivered, at least one surfactant and at least on dispersing agent applied to said outer surface of said tubular base in a first layer having a thickness from 0.3 mm to 3 mm, and a second layer of silicone rubber having a thickness from 0.02 to 1 mm covering said first layer on said tubular base whereby said drug will diffuse through said second layer when the device is inserted into the vagina at a more controlled rate over time.

2. The vaginal controlled drug delivery device in claim 1 wherein the drug is selected from group consisting of Mifepristone, Danazol, Pegesterone, Ralozifene, Tamoxifene and Nafoxidine.

3. The vaginal controlled drug delivery device in claim 1 wherein the surfactants are selected from one or more of the group consisting of Span 20-80, Brij 52-76, OP emulsifiers, Polyethylene glycol 400-20000, Pluronic-124, Pluronic-188, sodium lauryl sulphate, sodium teradecyl sulfate and Trolamine.

4. The vaginal controlled drug delivery device in claim 1 wherein the dispersing agent selected from one or more of the group consisting of glycerin, methylethylene glycol, succinic acid, colic acid, deoxycholic acid, hexadecyl alcohol, octadecul alcohol, B-type cyclodextrin, R Type cyclodextrin and silicone rubber.

5. The vaginal controlled drug delivery device in claim 1 wherein mixture comprises by weight 5 to 7 percent of the drug, 0.5 to 20% surfactants and from 74.5 to 94.5 percent.

6. The vaginal controlled drug delivery device in claim 1 wherein the silicone rubber is the second layer is selected from the group consisting of HTB, RTV-2, RTV-1, Dow Corning Medical Silicone Rubber Sitlastic-382, Dow Corning Medical Silicone Rubber Q7 and Dow Corning Implantable MDX Silicone Rubber.

7. The vaginal controlled drug device in claim 1 wherein the tubular base includes a hollow core.

8. The vaginal controlled drug delivery device in claim 1 wherein the tubular base is a ring.

9. The vaginal controlled drug delivery device in claim 1 wherein the tubular base is formed of silicone rubber selected from the group HTB, RTV-2, RTV-1, Dow Corning Medical Silicone Rubber Sitlastic-382, Dow Corning Medical Silicone Rubber Q7 and Dow Corning Implantable MDX Silicone Rubber.

10. A method of controlled vaginal delivery of drugs through gradual release comprising the steps of:
forming an inert base of a rubber composition;
applying a first layer on said inert base composed of a mixture of a drug to be administered, a surfactant and a dispersing agent, said first layer having a thickness less than 3 mm;
encapsulating said first layer with a second layer of s silicone rubber between said second layer and said inert base, said second layer having a thickness of less than 1 mm; and
inserting said device into the vaginal cavity of a patient to be treated with the drug whereby the dosage of the drug delivered is more uniformly controlled over time at higher concentrations.

11. The method defined in claim 10 wherein the drug is selected from group consisting of Mifepristone, Danazol, Pegesterone, Ralozifene, Tamoxifene and Nafoxidine.
